(19) United States
(12) Patent Application Publication
(10) Pub. No.: US 2024/0400490 A1
(43) Pub. Date: Dec. 5, 2024

(54) PROCESS FOR PREPARING A PEROXYESTER OR PEROXYCARBONATE

(71) Applicant: NOURYON CHEMICALS INTERNATIONAL B.V., Amsterdam (NL)

(72) Inventors: Waldo Joseph Elisabeth BEEK, Deventer (NL); Bjorn TER HORST, Maarn (NL)

(73) Assignee: NOURYON CHEMICALS INTERNATIONAL B.V., Amsterdam (NL)

(21) Appl. No.: 18/678,707

(22) Filed: May 30, 2024

(30) Foreign Application Priority Data
Jun. 1, 2023 (EP) 23176680.9

(51) Int. Cl. C07C 67/31; C07C 67/36; C07C 67/58; C07C 69/28

(52) U.S. Cl. CPC C07C 67/31; C07C 67/36; C07C 67/58; C07C 69/28

(57) ABSTRACT

A process for preparing a peroxyester or peroxycarbonate comprises the steps of:
reacting an organic hydroperoxide with an acid halide, an acid anhydride, or a haloformate, in the presence of a base to form an aqueous layer and an organic layer,
separating the aqueous layer from the organic layer after completion of step a),
adding a reducing agent to the organic layer after the aqueous layer has been separated from the organic layer in step b), wherein the reducing agent reduces the organic hydroperoxide to a corresponding alcohol and forms a mixture,
maintaining or adjusting the pH of the mixture of step c) to greater than about 6.8, and
maintaining the pH of the mixture of greater than about 6.8 for at least 5 seconds.

### CROSS-REFERENCE TO RELATED APPLICATION

This application claims the benefit of EP Patent Application Number EP23176680.9, filed Jun. 1, 2023, which is expressly incorporated herein by reference in its entirety.

### TECHNICAL FIELD

The present disclosure relates to a process for preparing a peroxyester or peroxycarbonate with improved storage stability and reduced hydroperoxide content.

### BACKGROUND

Peroxyesters and percarbonates find a wide variety of uses in the chemical field. They are typically produced by reacting an organic hydroperoxide with a reactive carbonyl species, such as an acid halide, an acid anhydride, or a haloformate.

An issue with peroxyesters and percarbonates is that they often include undesirably high levels of hydroperoxide, which can be problematic from a regulatory and/or application viewpoint.

Additionally, peroxyesters and percarbonates often have poor storage stability.

The standard workup process for this reaction comprises one or more washing steps in an attempt to reduce the residual organic hydroperoxide present in an organic layer (see e.g., EP1382596). In CN112300044 the reaction workup was performed at pH 7.5-10, and yields a poor product (see Comparative Example 5 below). In addition, other objects, desirable features and characteristics will become apparent from the subsequent summary and detailed description, and the appended claims, taken in conjunction with the accompanying drawings and this background.

### BRIEF SUMMARY

This disclosure provides a process for preparing a peroxyester or peroxycarbonate comprising:
a) reacting an organic hydroperoxide with an acid halide, an acid anhydride, or a haloformate, in the presence of a base to form an aqueous layer and an organic layer,
b) separating the aqueous layer from the organic layer after completion of step a),
c) adding a reducing agent to the organic layer after the aqueous layer has been separated from the organic layer in step b), wherein the reducing agent reduces the organic hydroperoxide to a corresponding alcohol and forms a mixture,
d) maintaining or adjusting the pH of the mixture of step c) to greater than about 6.8, and
e) maintaining the pH of the mixture of greater than about 6.8 for at least 5 seconds,
wherein
i. the mixture of step c) has a pH of less than about 6.8 before step d), and in step d) the pH of the mixture of step c) is increased to a pH of greater than about 6.8; and/or
ii. after completion of step e) the pH of the mixture is decreased to a pH of less than about 6.8.

It was unexpectedly found that the hydroperoxide content in the peroxide product could be substantially reduced whilst simultaneously giving rise to a peroxy product with much improved storage stability by a specific process that manipulates the reaction mixture pH during the initial workup process.

### DETAILED DESCRIPTION

The following detailed description is merely exemplary in nature and is not intended to limit the present disclosure or the application and uses of the present disclosure. Furthermore, there is no intention to be bound by any theory presented in the preceding background of the present disclosure or the following detailed description. Moreover, it is contemplated that, in various non-limiting embodiments, it is to be appreciated that all numerical values as provided herein, save for the actual examples, are approximate values with endpoints or particular values intended to be read as “about” or “approximately” the value as recited.

In a first aspect, the present disclosure relates to a process for preparing a peroxyester or peroxycarbonate comprising:
a) reacting an organic hydroperoxide with an acid halide, an acid anhydride, or a haloformate, in the presence of a base,
b) separating the aqueous layer after completion of step a),
c) adding a reducing agent to the organic layer after the aqueous layer has been separated in step b), wherein the reducing agent is an agent capable of reducing the organic hydroperoxide to the corresponding alcohol,
d) ensuring that the mixture of step c) has a pH of greater than 6.8, typically greater than 7.0, typically greater than 7.2, and more typically greater than 7.4, by maintaining or increasing the pH of the mixture of step c), and
e) maintaining the pH of greater than 6.8, typically greater than 7.0, typically greater than 7.2, and more typically greater than 7.4, for at least 5 seconds, typically at least 10 seconds, typically at least 30 seconds, typically at least 60 seconds, and more typically at least 120 seconds,
wherein
   i. the mixture of step c) has a pH of less than 6.8 before step d), typically a pH of about 6.5 or lower, and most typically a pH within the range of about 4 to 6.5, and in step d) the pH of the mixture of step c) is increased to a pH of greater than 6.8, typically greater than 7.0, typically greater than 7.2, and more typically greater than 7.4; and/or
   ii. after completion of step e) the pH is decreased to a pH of less than 6.8, typically to a pH of about 6.5 or lower, and most typically to a pH within the range of about 4 to 6.5.

Steps a) and b) follow the normal process for producing peroxyesters or peroxycarbonates, wherein the organic hydroperoxide is reacted with an acid halide, an acid anhydride, or a haloformate, in the presence of a base. The equivalent amount of the reactive carbonyl compound (acid halide, acid anhydride, or haloformate) relative to the organic hydroperoxide is not particularly limited, but typically is in the range of about 0.25-5 equivalents, typically in the range of about 0.6-1.1 equivalents, more typically in the range of about 0.6-1 equivalents, and most typically in the range of about 0.8-1 equivalents.

The reaction conditions of step a) are conventional. The temperature generally is in the range of −10° C. to 70° C. and suitably between 0° C. to 50° C. The pH is basic, i.e., above 7. Generally, the pH is in the range of 9-14. In practice, the pH is above 10, and the common pH range is from 11 to 13.5. The reaction may proceed under ambient pressure and in free contact with the atmosphere. Any suitable base may be used to adjust the pH to a basic pH, such as, but not limited to, sodium carbonate, potassium carbonate, sodium hydroxide, potassium hydroxide, and mixtures thereof. Such bases are typically used in the form of aqueous solutions thereof. The reaction mixture may also comprise conventional auxiliaries, such as NaCl(aq.) to assist phase separation (for step b)). The reaction can be performed neatly or in the presence of solvent(s).

The organic hydroperoxide may be an organic hydroperoxide of the general formula (II):

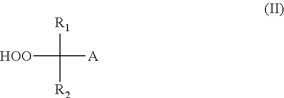

wherein:
   R₁ and R₂ are independently chosen from hydrogen, C₁-C₂₀ alkyl, C₃-C₂₀ cycloalkyl, C₆-C₂₀ aryl, C₇-C₂₀ aralkyl, and C₇-C₂₀ alkaryl, or R₁ and R₂ form a C₃-C₁₂ cycloalkyl group, which groups may include linear or branched alkyl moieties, and each of R₁ and R₂ may optionally be substituted with one or more groups selected from hydroxy, hydroperoxy, alkoxy, linear or branched alkyl, aryloxy, halogen, ester, carboxy, nitrile, and amido groups,
   A is selected independently of R₁ and R₂ from the same group of substituents as R₁ and R₂, or
   A is of the general formula (III):

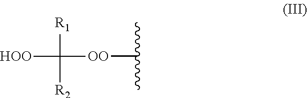

wherein R₁ and R₂ are as defined above, wherein A is typically selected independently of R₁ and R₂ from the same group of substituents as R₁ and R₂.

Typical organic hydroperoxides include tert-butyl hydroperoxide, tert-amyl hydroperoxide, cumyl hydroperoxide, 1,1,3,3-tetramethylbutyl hydroperoxide, 4-hydroperoxy-4-methylpentan-2-ol, 2,5-dihydroperoxy-2,5-dimethylhex-3-yne, 2,5-dihydroperoxy-2,5-dimethylhexane, or mixtures thereof. The most typical organic hydroperoxide for use in this process is tert-butyl hydroperoxide (TBHP), typically tert-butyl hydroperoxide obtained from an air oxidation process.

The acid halide, acid anhydride, or haloformate may be a reactive carbonyl compound of the general formulae (Ia) or (Ib)

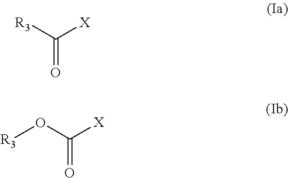

wherein:
   R₃ is independently chosen from C₁-C₂₀ alkyl, C₃-C₂₀ cycloalkyl, C₆-C₂₀ aryl, C₇-C₂₀ aralkyl, and C₇-C₂₀ alkaryl, wherein R₃ is optionally substituted with one or more groups selected from hydroxy, alkoxy, linear or branched alkyl, aryloxy, halogen, ester, carboxy, nitrile, and amido groups, and
   X is a halide (typically chloride) or —O—CO—R₃, wherein R₃ is selected independently of R₃ from the same group of substituents as R₃.

Typically, the acid halide or acid anhydride is derived from any one or more of the following carboxylic acids: acetic acid, phenylacetic acid, phenoxyacetic acid, propanoic acid, isobutyric acid, n-butyric acid, benzoic acid, 2-methyl-benzoic acid, 2-methylbutanoic acid, 2-butenoic acid, 3-phenylpropenic acid, 2,2-dimethylpropanoic acid, 2,2-dimethylbutanoic acid, 2,2-dimethylpentanoic acid, 2-ethylbutanoic acid, 3,5,5-trimethylhexanoic acid, 2-ethylhexanoic acid, neohexanoic acid, neoheptanoic acid, neodecanoic acid, octanoic acid, nonanoic acid, lauric acid, hexanedioic acid, 3,5,5-trimethylhexanedioic acid, 2,4,4-trimethylhexanedioic acid, decanedioic acid, undecanedioic acid, dodecanedioic acid, cyclohexanecarboxylic acid, 1,4-cyclohexanedicarboxylic acid, cyclohexane-1,4-diacetic acid, maleic acid, citric acid, methylsuccinic acid, citraconic acid, fumaric acid, oxalic acid, terephthalic acid, propenoic acid, and phthalic acid.

Typically, the process uses an acid halide or a haloformate.

Typically, the acid halide is an acid chloride. Typically, the acyl portion of the acid chloride corresponds to the acyl portion of any of the following carboxylic acids: acetic acid, phenylacetic acid, phenoxyacetic acid, propanoic acid, isobutyric acid, n-butyric acid, benzoic acid, 2-methyl-benzoic acid, 2-methylbutanoic acid, 2-butenoic acid, 3-phenylpropenic acid, 2,2-dimethylpropanoic acid, 2,2-dimethylbutanoic acid, 2,2-dimethylpentanoic acid, 2-ethylbutanoic acid, 3,5,5-trimethylhexanoic acid, 2-ethylhexanoic acid, neohexanoic acid, neoheptanoic acid, neodecanoic acid, octanoic acid, nonanoic acid, lauric acid, hexanedioic acid, 3,5,5-trimethylhexanedioic acid, 2,4,4-trimethylhexanedioic acid, decanedioic acid, undecanedioic acid, dodecanedioic acid, cyclohexanecarboxylic acid, 1,4-cyclohexanedicarboxylic acid, cyclohexane-1,4-diacetic acid, maleic acid, citric acid, methylsuccinic acid, citraconic acid, fumaric acid, oxalic acid, terephthalic acid, propenoic acid, and phthalic acid.

Typically, the haloformate is a chloroformate. Typical haloformates include 2-(1-methylethoxy)phenyl chloroformate, 1-methylpropyl chloroformate, 4-methylphenyl chloroformate, heptyl chloroformate, cyclohexyl methyl chloroformate, ethylene glycol bis(chloroformate), phenyl chloroformate, 3-methoxybutyl chloroformate, 2-phenoxyethyl chloroformate, 2,2-dimethyl-1,3-propane diol bis(chloroformate), phenyl methyl chloroformate, 9-octadecenyl chloroformate, 2-methylphenyl chloroformate, bisphenol A bis(chloroformate), 1,3-dimethyl butyl chloroformate, 3,4-dimethyl butyl chloroformate, 3,4-dimethyl phenyl chloroformate, 1,4-butane diol bis(chloroformate), 1,1-bis (ethoxycarbo)ethyl chloroformate, 3,5-dimethyl phenyl chloroformate, octyl chloroformate, ethyl chloroformate, octadecyl chloroformate, (2-oxo-1,3-dioxolan-4-yl)methyl chloroformate, 1,6-hexane diol bis(chloroformate), 2-chlorobutyl chloroformate, 4-methoxyphenyl chloroformate, 2-methylpropyl chloroformate, dodecyl chloroformate, 1,4-cyclohexane dimethanol bis(chloroformate), 2-chloro-2-phenyl ethyl chloroformate, 2-acryloyloxyethyl chloroformate, 4-nitrophenyl chloroformate, n-butyl chloroformate, decyl chloroformate, 2-ethylhexyl chloroformate, 2-propenyl chloroformate, 2-chlorocyclohexyl chloroformate, 2-methyl-2-propenyl chloroformate, cyclohexyl chloroformate, 2-chloroethyl chloroformate, [4-(phenylazo)phenyl]methyl chloroformate, hexadecyl chloroformate, 1-naphthalenyl chloroformate, chloroformate, 3,5,5-trimethyl hexyl chloroformate, isotridecyl chloroformate, tridecyl chloroformate, 4-(1,1-dimethylethyl)cyclohexyl chloroformate, chloroformate, 3-chloropropyl chloroformate, tetradecyl chloroformate, chloroformate, methyl chloroformate, 2-(1-methylethyl)phenyl chloroformate, triethylene glycol bis(chloroformate), 2-methoxyethyl chloroformate, 1-methylethenyl chloroformate, 3-methylphenyl chloroformate, 2 chloroformate, diethylene glycol bis(chloro-formate), 3-methyl-5-(1-methylethyl)phenyl chloroformate, 2-ethoxyethyl chloroformate, 3-methyl-1,5-pentane diolbis(chloroformate), 4-methoxy carbophenyl chloroformate, ethenyl chloroformate, 1-methylethyl chloroformate, 2-(1-methylpropyl)phenyl chloroformate, chloroformate, pentyl chloroformate, cyclodecyl chloroformate, 4-(1,1-dimethylethyl)phenyl chloroformate, hexyl chloroformate, n-propyl chloroformate, 3-methoxy-3-methylbutyl chloroformate, 2-propoxyethyl chloroformate, 2-methoxy-1-methylethyl chloroformate, 2-butoxyethyl chloroformate, 2,2-dimethyl propyl chloroformate, 2,3-dihydro-2,2-dimethyl-7-benzofuranyl chloroformate, 1-chloroethyl chloroformate, cyclobutyl chloroformate, 5-methyl-2-(1-methylethyl)cyclohexyl chloroformate, 1,1-dimethyl ethyl chloroformate, 1-methylheptyl chloroformate, and mixtures thereof.

After completion of step a), the reaction mixture is typically allowed to settle, thereby forming an aqueous layer and an organic layer (i.e., two separate/immiscible phases). The aqueous layer that forms upon allowing the mixture to settle is separated in step b). The organic layer that remains is taken through to step c).

In step c), the reducing agent is added to the organic layer. Typically, the reducing agent is a sulfur based reducing agent, such as a dithionite, hydrosulfite, metabisulfite, sulfide or a sulfite. The reducing agent serves to reduce any remaining hydroperoxide to the corresponding alcohol. Typical sulfites include metal sulfites, metal bisulfites, and/or metal metabisulfites. Typical metals include the alkali and alkaline metals, such as sodium or potassium. Typically, the reducing agent added in step c) is sodium metabisulphite and/or sodium sulfite, typically sodium metabisulphite. The reducing agent is typically added in step c) as an aqueous solution of the reducing agent. As such, in a most typical embodiment, the reducing agent added in step c) is an aqueous solution of sodium metabisulphite. The reducing agent (RA) to hydroperoxide (HP) molar ratio (RA:HP) is typically >1:1, typically >1:1 to about 5:1, typically >1:1 to about 3:1, more typically >1:1 to about 2:1, and most typically about 1.3:1 to about 1.8:1. It is typical if the reaction mixture is agitated (e.g., stirred) for at least about 1 minute (e.g., about 1-10 minutes) before proceeding to the next step of the process.

In a typical embodiment (step i)), the pH of the mixture of step c) is lowered to a pH of less than 6.8 before proceeding to step d), typically to a pH of about 6.5 or lower, and most typically to a pH within the range of about 4 to 6.5. The pH drop may be achieved by any conventional techniques, such as by adding an acid, e.g., H2SO4(aq.), by adding a buffer with a pH of less than 6.8, and/or by using a reducing agent with a pH of less than 6.8. The pH of the mixture of step c) may be decreased as a consequence of dosing the reducing agent to the organic layer (typical reducing agents, such as aqueous sulfite solutions, can be weakly acidic, so dosing those acidic reducing agents to the organic layer in step c) may result in the mixture having a pH of <6.8). Typically, a buffer with a pH in the range of about pH 4-6.5 is used to effect the pH drop. Typical buffers include acetate buffer with a pH of from about 4 to 6.5 (other suitable buffers include, but are not limited to, citrate acid buffer or monopotassiumphosphate buffer). The mixture is typically agitated (e.g., stirred) at this reduced pH for at least about 1 minute (e.g., about 1 to about 25 minutes) before proceeding to the next step of the process.

Step d) typically ensures that the pH of the mixture of step c) is at a pH of greater than 6.8, typically greater than 7.0, typically greater than 7.2, and more typically greater than 7.4. For example, the pH may be >8. This is achieved either by maintaining the pH of the mixture (if the mixture of step c) is already at a pH of >6.8) or by increasing the pH of the mixture to a pH of greater than 6.8 (particularly if the pH of the mixture of step c) is adjusted to a pH of less than 6.8 before step d)). A pH increase may be achieved by adding a base in an amount necessary to reach the desired pH value. Suitable bases include, but are not limited to, sodium carbonate, potassium carbonate, sodium hydroxide, potassium hydroxide, and mixtures thereof. Such bases are typically used in the form of aqueous solutions thereof.

In step e), the pH of greater than 6.8, typically greater than 7.0, typically greater than 7.2, and more typically greater than 7.4, is maintained for at least 5 seconds, typically at least 10 seconds, typically at least 30 seconds, typically at least 60 seconds, and more typically at least 120 seconds, such as from about 120 seconds to about 600 seconds. The mixture is typically agitated (e.g., stirred) during step e).

In a typical embodiment, the process further comprises step f) (step f)=step ii)), wherein after completion of step e) the pH of the mixture is subsequently decreased to a pH of less than 6.8, typically to a pH of about 6.5 or lower, and most typically to a pH within the range of about 4 to 6.5. The pH drop may be achieved by any conventional techniques, such as by adding an acid, e.g., H2SO4(aq.) or by adding a buffer with a pH of less than 6.8. Typically, a buffer with a pH in the range of about pH 4-6.5 is used to effect the pH drop. Typical buffers include acetate buffer with a pH of from about 4 to 6.5 (other suitable buffers include, but are not limited to, citrate acid buffer or monopotassium phosphate buffer). The pH of the mixture of step f) is typically maintained for at least 5 seconds, typically at least 10 seconds, typically at least 30 seconds, typically at least 60 seconds, such as from about 90 seconds to about 600 seconds, during which time the mixture is typically agitated (e.g., stirred). Optionally, at the end of step f) the pH may be increased to a pH of greater than 6.8 (i.e., step d) may be optionally repeated at the end of step f)).

The aqueous layer of step f) (that forms after the mixture is allowed to settle into an aqueous layer and an organic layer, i.e., two immiscible phases) is separated, and the remaining organic layer may be subjected to one or more washing steps g) (to remove any residual water-soluble impurities, such as excess sulfite, alcohol and sulfate generated during the reaction). The washing step(s) may comprise one or more water washes and/or one or more alkaline aqueous washes (e.g., washing with NaOH(aq) and/or NaHCO3(aq)). The washing step(s) typically comprises one or more neutral to alkaline aqueous washes. For the avoidance of doubt, the peroxyester or peroxycarbonate is present in this organic layer. The final organic layer may be dried using conventional techniques, such as drying agents (e.g., MgSO4), vacuum, and/or air drying.

It is preferable for the temperature of the mixture during steps c)-g) to be maintained at about 0-70° C., more typically about 0-40° C.

In a typical embodiment, the present disclosure relates to a process for preparing a peroxyester or peroxycarbonate comprising:
a) reacting an organic hydroperoxide with an acid halide, an acid anhydride, or a haloformate, in the presence of a base,
b) separating the aqueous layer after completion of step a),
c) adding a reducing agent to the organic layer after the aqueous layer has been separated in step b), wherein the reducing agent is an agent capable of reducing the organic hydroperoxide to the corresponding alcohol, and wherein the mixture of the organic layer and the reducing agent has a pH of less than 6.8, typically a pH of about 6.5 or lower, and most typically a pH within the range of about 4 to 6.5,
d) increasing the pH of the mixture of step c) to a pH of greater than 6.8, typically greater than 7.0, typically greater than 7.2, and more typically greater than 7.4,
e) maintaining the pH of greater than 6.8, typically greater than 7.0, typically greater than 7.2, and more typically greater than 7.4, for at least 5 seconds, typically at least 10 seconds, typically at least 30 seconds, typically at least 60 seconds, and more typically at least 120 seconds,
f) decreasing the pH after completion of step e) to a pH of less than 6.8, typically to a pH of about 6.5 or lower, and most typically to a pH within the range of about 4 to 6.5, and
g) optionally repeating step d) (i.e., increase the pH of the mixture of step f) to a pH or greater than 6.8), and/or optionally washing the organic layer of step f), wherein the optional washing step(s) typically comprise one or more water washes and/or one or more alkaline aqueous washes (e.g., washing with NaOH(aq) and/or NaHCO₃(aq)).

This process is particularly relevant for processes which use hydroperoxides manufactured by air oxidation (notably t-butyl hydroperoxide). In that respect, a surprising observation was that, using the typical processes disclosed herein, it was possible to consistently produce peroxyesters and peroxycarbonates from t-butyl hydroperoxide that have an initial (t=0 weeks) tert-butylhydroperoxide (TBHP) content of less than 300 ppm and a four-week stability (0-4 week ΔTBHP) value of 300 ppm or less (the initial TBHP content and the four-week stability value are determined in accordance with the “TBHP Protocol” described in the Worked Examples below). This very high product purity and stability profile was unexpected.

Accordingly, in another aspect, the present disclosure relates to a tert-butyl peroxyester or a tert-butyl peroxycarbonate, typically a tert-butyl peroxyester, exemplified in that the tert-butyl peroxyester or tert-butyl peroxycarbonate has an initial (t=0 weeks) tert-butylhydroperoxide (TBHP) content of less than 300 ppm and a four-week stability (0-4 week ΔTBHP) value of 300 ppm or less, wherein the initial TBHP content and the four-week stability value are determined in accordance with the “TBHP Protocol” as set out in the worked examples below. For the avoidance of doubt a “tert-butyl peroxyester” is a peroxyester that is obtainable by reacting TBHP with an acid halide or an acid anhydride (i.e., t-Bu-OO—C(O)R), and a “tert-butyl peroxycarbonate” is a peroxycarbonate that is obtainable by reacting TBHP with a haloformate (i.e., t-Bu-OO—C(O)OR). Non-limiting examples of tert-butyl peroxyesters include tert-butyl peroxy-3,5,5-trimethyl hexanoate (CAS: 13122-18-4), tert-Butyl peroxy-2-ethylhexanoate (CAS: 3006-82-4), and tert-Butyl peroxybenzoate (CAS: 614-45-9).

It has also been found that this process is particularly effective for preparing peroxyesters or percarbonates that do not include any free acid groups. Accordingly, in a typical embodiment, the process disclosed herein is for preparing a peroxyester or peroxycarbonate, wherein the peroxyester or peroxycarbonate includes no free acid groups.

### EXAMPLES

The following examples provide a detailed method for working the present disclosure. These worked examples are exemplary in nature and not intended to be limiting.

### A. Preparation of tert-butyl peroxy-3,5,5-trimethyl hexanoate (CAS: 13122-18-4) [Steps a) & b)]

To a 1 L jacketed glass reactor, equipped with baffles, a pH electrode, an overhead mechanical stirrer (pitch blade agitator, size ⅓ of the diameter of the reactor) and glycol/water temperature control, 148.4 g TBHP (70 wt % in water) was added. The reaction medium was cooled to 10° C. under agitation (1000 rpm) and 125.8 g NaOH-25 solution (25 wt % NaOH in water) was added in 20 min. After the addition, agitation was increased to 1300 rpm and 93.6 gram Isononanoyl chloride was dosed in 16 min and the temperature was allowed to rise to 25° C. and maintained at that temperature. In a second addition step 93.5 g isononanoyl chloride and 60.4 g NaOH-25 were added simultaneously in 30 min during which the temperature was allowed to rise to 30° C. and maintained at that temperature. After the second addition the reaction mixture was stirred for 9 min at 30° C. and 1300 rpm. The reaction mixture was quenched with 75 g demineralized water and stirred for 1 min. Agitation was stopped and the organic phase and water phase were allowed to separate. The water phase was drained from the reactor and discarded.

Reducing Agent Addition Step [Step c)]

Agitation was restarted (1000 rpm) and 5.0 g buffer solution (16.3 wt % AcOH, 62.5 wt % water and 21.2 wt % NaOH-25) was added followed by the addition of 105 gram water. The temperature was set to 25° C. by adjusting the jacketed temperature. The pH was adjusted to pH=5.5 by addition of a few drops of NaOH-25. To the buffered solution 55.5 grams of a freshly prepared sulfite solution (69 wt % water, 18 wt % sodium metabisulfite Na2S2O5 (s) and 13 wt % NaOH-25) was added dropwise over a period of 20 minutes. During the dosing a pH of 5.5 was maintained by the addition of NaOH-25 (approximately 2 grams in total).

### Example 1 [Step i)—the Mixture of Step c) has a pH of Less than 6.8 Before Proceeding to Step d)]

After the Addition of the Sulfite Solution, the Reaction Mixture was Stirred for 22 Minutes at a pH of 5.5. The pH of the reaction mixture was then increased to pH 8.0 by the dropwise addition of NaOH-25 (approximately 1 g over 5 minutes) (steps d) and e)), followed by workup (see below).

### Example 2 [Step i)—the Mixture of Step c) has a pH of Less than 6.8 Before Proceeding to Step d); and Step ii)—after Completion of Step e) the pH is Decreased to a pH of Less than 6.8]

After the addition of the sulfite solution, the reaction mixture was stirred for 5 minutes at a pH of 5.5. The pH was then increased to pH 10 by the dropwise addition of 9.0 g NaOH-25 over 2 minutes (step d)). The mixture was stirred for 3 minutes at pH 10 (step e)). The pH was then lowered back to 5.5 by the dropwise addition of 4 g HCl (15 wt % HCl in water) over 10 minutes (step ii)). The reaction mixture was stirred for 2 minutes at a pH of 5.5. Total reduction time was 22 minutes (cf. Example 1). The pH of the reaction mixture was then increased to pH 8.0 by the dropwise addition of NaOH-25 (approximately 1 g over 5 minutes), followed by workup (see below)

### Workup for Examples 1 and 2

Agitation was stopped and the organic phase and water phase were allowed to separate. The water phase was drained from the reactor and discarded. Next, 55 g NaCl-25 (25 wt % NaCl in water), 180 g demineralized water and 28 g NaHCO₃-6 (6 wt % NaHCO3 in water) were added to the remaining organic layer. After the addition the mixture was stirred (1000 rpm) at 25° C. for 3 minutes. Agitation was stopped and the organic phase and water phase were allowed to separate. The water phase was drained from the reactor and discarded. Subsequently the organic phase was drained and collected separately in a 500 ml Erlenmeyer flask. The organic phase was dried over 10 grams MgSO4.2H2O for 10 minutes. The mixture was filtered over a glass filter and the clear organic peroxide product 218 gram (assay 99.4%) was collected and stored at 20° C.

### TBHP Content Determination in Final Product (“TBHP Protocol”)

The following reagents and apparatus were used in the determination of the TBHP content of the peroxide product:
Solution A: Sulphur dioxide solution approx. 0.005 mol/L SO₂ solution in methanol
Solution B: Methyl ethyl ketone 1000 mL with approximately 15 μL tert. Butyl hydroperoxide (70 wt %) and 50 mL water.
Equipment: Potentiometric titrator Metrohm combined Pt Titrode (art. 6.0431.100). Conditions of Metrohm Titrando 888 are:
Monotone titration
Start volume: 0.1 mL
Volume increment: 0.07 mL
Dosing rate: 2 mL/minute
Minimal waiting time: 0 seconds
Maximum waiting time: 4 seconds

The TBHP content of the final peroxide product was determined by dissolving 1 g of peroxide product in 25 mL Solution B. The solution was titrated immediately with the Sulphur dioxide solution A until slightly beyond the potential jump. The measurement was performed in duplicate. Two blanks were also performed, and the average blank was calculated. The following formula was used to calculate the hydroperoxide content (TBHP):

$$Hydroperoxide content ( mg / kg ) = \frac{( {V}_{1} - {V}_{0} ) \cdot c \cdot Mm \cdot 1000}{m}$$

Where:
V₁ mL of sulphur dioxide solution to titrate the sample
V₀ mL of sulphur dioxide to titrate the blank
e molarity of the sulphur dioxide solution
Mm molar mass of the hydroperoxide concerned, for bi functional hydroperoxides use
m mass of sample in grams

### Results:

| | TBHP | TBHP | TBHP | TBHP | ΔTBHP* | ΔTBHP* |
|---|---|---|---|---|---|---|
| | (ppm) | (ppm) | (ppm) | (ppm) | (ppm) | (ppm) |
| | t = 0 | t = 2 | t = 4 | t = 8 | 0-4 | 0-8 |
| | weeks | weeks | weeks | weeks | weeks | weeks |
| Example 1 | 1324 | 1663 | 1696 | 1732 | 372 | 408 |
| Example 2 | 179 | 330 | 364 | 436 | 185 | 257 |

*A lower ΔTBHP value indicates increased storage stability

For the avoidance of any doubt, the term “four-week stability (0-4 weeks ΔTBHP) value” as used herein means the difference in TBHP, in ppm, between the TBHP value measured at week 0 (i.e., the initial TBHP value) and the TBHP value measured after a storage period of four weeks, wherein both the week 0 and week 4 values are determined in accordance with the above “TBHP protocol”. For the purposes of the present disclosure, the “four-week stability (0-4 weeks ΔTBHP) value” is determined by storing the peroxide for a period of 4 weeks at 20° C.

### B. Preparation of tert-Butyl peroxy-2-ethylhexanoate (CAS: 3006-82-4) [Steps a) and b)]

To a 1 L jacketed glass reactor, equipped with baffles, a pH electrode, an overhead mechanical stirrer (pitch blade agitator, size ⅓ of the diameter of the reactor) and glycol/water temperature control, 177.3 g TBHP (70 wt % in water) was added. The reaction medium was kept at 25° C. under agitation (1000 rpm) and 138.3 g NaOH-25 solution was added in 20 min. After the addition, agitation was increased to 1300 rpm and 204.2 g 2-ethyl hexanoyl chloride and 88.4 g NaOH-25 were added simultaneously over 35 minutes during which the temperature was allowed to rise to 40° C. and maintained at that temperature. After the addition the reaction mixture was stirred for an additional 45 minutes at 40° C. and 1300 rpm. The reaction mixture was cooled to 20° C. and 9.0 g NaOH-25 was added. The reaction mixture was stirred for 2 minutes. The reaction mixture was quenched with 72.0 g demineralized water and stirred for 1 min. Agitation was stopped and the organic phase and water phase were allowed to separate. The water phase was drained from the reactor and discarded.

Reducing Agent Addition Step [Step c)]

To the unstirred reactor content, 3.4 g buffer solution (16.3 wt % AcOH, 62.5 wt % water and 21.2 wt % NaOH-25) were added followed by the addition of 100 g water. The temperature was kept at 20° C. by jacketed temperature control. The pH was adjusted to pH 5.5 by addition of a few drops of NaOH-25. To the buffered solution 56 g of a freshly prepared sulfite solution (69 wt % water, 18 wt % sodium metabisulfite Na2S2O5 (s) and 13 wt % NaOH-25) was added dropwise over a period of 6 minutes. During the dosing a pH of 5.5 was maintained by the addition of NaOH-25 (approximately 7 g in total).

### Example 3 [Step i)—the Mixture of Step c) has a pH of Less than 6.8 Before Proceeding to Step d)]

After the addition of the sulfite solution, the reaction mixture was stirred for 20 minutes at a pH of 5.5. The pH of the reaction mixture was then increased to 8.0 by the dropwise addition of approximately 1.0 gram NaOH-25 in 5 minutes (steps d) and e)), followed by workup (see below).

### Example 4 [Step i)—the Mixture of Step c) has a pH of Less than 6.8 Before Proceeding to Step d); and Step ii)—after Completion of Step e) the pH is Decreased to a pH of Less than 6.8]

After the addition of the sulfite solution the reaction mixture was stirred for 5 minutes at a pH of 5.5. The pH was then increased to 10 by the dropwise addition of 8.4 g NaOH-25 over 5 minutes (step d)). The mixture was stirred for 3 minutes at a pH of 10 (step e)). The pH was lowered back to 5.5 by the dropwise addition of 5.4 g HCl (15 wt % in water) over 5 minutes (step ii)). The reaction mixture was stirred for 2 minutes at a pH of 5.5. Total reduction time was 20 minutes (cf. Example 3). The pH of the reaction mixture was then increased to 8.0 by the dropwise addition of approximately 1.0 gram NaOH-25 in 5 minutes, followed by workup (see below).

### Workup for Examples 3 and 4

Agitation was stopped and the organic phase and water phase were allowed to separate. The water phase was drained from the reactor and discarded. Next, 33.0 gram NaCl-25 (25 wt % in water), 102 gram demineralized water and 36.3 gram NaHCO3-6 (6 wt % NaHCO₃ in water) were added to the unstirred reaction mixture. After the addition the reaction mixture was stirred (1000 rpm) at 20° C. for 2 minutes. Agitation was stopped and the organic phase and water phase were allowed to separate. The water phase was drained from the reactor and discarded. Subsequently the organic phase was drained and collected separately in a 500 ml Erlenmeyer. The organic phase was dried over 10 grams MgSO4.2H2O for 10 minutes. The mixture was filtered over a glass filter and the clear organic peroxide product 246 gram (assay 98.8%) was collected and stored at 4° C.

Analysis: Examples 3 and 4 were analyzed using the same method as is described for Examples 1 and 2 (“TBHP Protocol”).

| | TBHP (ppm) | TBHP (ppm) | TBHP (ppm) | ΔTBHP (ppm)* |
|---|---|---|---|---|
| | t = 0 weeks | t = 2 weeks | t = 4 weeks | 0-4 weeks |
| Example 3 | 5 | 71 | 105 | 100 |
| Example 4 | 0 | 45 | 75 | 75 |

*A lower ΔTBHP value indicates increased storage stability.

### C. Preparation of tert-Butyl peroxybenzoate (CAS: 614-45-9) [Steps a) and b)]

To a 1 L jacketed glass reactor, equipped with baffles, a pH electrode, an overhead mechanical stirrer (pitch blade agitator, size ⅓ of the diameter of the reactor) and glycol/water temperature control, 55.9 g NaCl-25% (aq.) and 160.0 g TBHP (70 wt % in water) were added. The reaction medium was kept at 20° C. under agitation (1000 rpm) and 59.9 g NaOH-25 solution was added in 5 minutes. After the addition, 57.4 g benzoyl chloride was added in 14 minutes during which the temperature was maintained at 20° C. Next, 108.0 g benzoyl chloride and 131.9 g NaOH-25 solution were added simultaneously over 19 minutes during which the was maintained at 20° C. After the addition the reaction mixture was stirred for an additional 32 minutes at 20° C. and 1100 rpm. After the post reaction 20.0 g NaOH-25 was added and the mixture was stirred for an additional 5 minutes. Agitation was stopped, and the organic phase and water phase were allowed to separate. The water phase was drained from the reactor and discarded.

### Reducing Agent Addition Step [Step C]

Examples 5 and 6: To the unstirred reactor content, 45 g water and 118 g NaCl-25 (aq) were added, and the agitator was restarted (1100 RPM). The pH was adjusted to pH 10 with 6.2 g NaOH-25. The temperature was kept at 20° C. by jacketed temperature control. To the stirred solution 46 g freshly prepared sulfite solution (69 wt % water, 18 wt % sodium metabisulfite Na2S2O5 (s) and 13 wt % NaOH-25) was added dropwise over a period of 6 minutes. During the dosing a pH of 10 was maintained by the addition of NaOH-25 (approximately 8.1 g in total).

Examples 7 and 8: To the unstirred reactor content, 45 g water, 118 g NaCl-25 (aq.) and 10 g buffer solution (16.3 wt % AcOH, 62.5 wt % water and 21.2 wt % NaOH-25) were added. The temperature was kept at 20° C. by jacketed temperature control. The pH was adjusted to pH 5.5 by addition of a few drops of NaOH-25. To the buffered solution 46 g of a freshly prepared sulfite solution (69 wt % water, 18 wt % sodium metabisulfite Na2S2O5 (s) and 13 wt % NaOH-25) was added dropwise over a period of 5 minutes. During the dosing a pH of 5.5 was maintained by the addition of NaOH-25 (approximately 2 g in total).

### Example 5 [Comparative]

The reaction mixture was stirred for an additional 30 minutes after the sulfite solution was dosed and the pH was maintained at pH 10, followed by workup (see below).

### Example 6 [Step i)—the Mixture of Step c) has a pH of Less than 6.8 Before Proceeding to Step d)]

The reaction mixture was stirred for an additional 25 minutes after the sulfite solution was dosed and the pH was maintained at pH 10. The pH was subsequently lowered to pH 6.4 by the addition of 6.2 g HCl (15 wt % in water) in 3 minutes (step i)). The reaction mixture was stirred for 2 minutes at pH of 6.4. (total time 30 min; cf. Comparative Example 5). The pH was then brought to 10 with the addition of NaOH-25 (steps d) and e)), followed by workup (see below).

### Example 7 [Step i)—the Mixture of Step c) has a pH of Less than 6.8 Before Proceeding to Step d)]

After the addition of the sulfite solution, the reaction mixture was stirred for 20 minutes at a pH of 5.5. The pH of the reaction mixture was then increased to 7.6 by the dropwise addition of approximately 5 g NaOH-25 in 5 minutes (steps d) and e)), followed by workup (see below).

### Example 8 [Step i)—the Mixture of Step c) has a pH of Less than 6.8 Before Proceeding to Step d); and Step ii)—After Completion of Step e) the pH is Decreased to a pH of Less than 6.8]

After the addition of the sulfite solution the reaction mixture was stirred for 5 minutes at a pH of 5.5. The pH was then increased to 10 by the dropwise addition of 7 g NaOH-25 over 5 minutes (step d)). The mixture was stirred for 3 minutes at a pH of 10 (step e)). The pH was lowered back to 5.5 by the dropwise addition of 9.6 g HCl (15 wt % in water) over 5 minutes (step ii)). The reaction mixture was stirred for 2 minutes at a pH of 5.5. Total reduction time was 20 minutes (cf. Example 7). The pH of the reaction mixture was then increased to 7.6 by the dropwise addition of approximately 5 g NaOH-25 in 5 minutes, followed by workup (see below).

### Workup for Examples 5-8

Agitation was stopped and the organic phase and water phase were allowed to separate. The water phase was drained from the reactor and discarded. Next, 100 g NaCl-25 (25 wt % in water) and 45 g demineralized water were added to the unstirred reaction mixture. After the addition the reaction mixture was stirred (1000 rpm) at 20° C. for 2 minutes. Agitation was stopped and the organic phase and water phase were allowed to separate. The water phase was drained from the reactor and discarded. Subsequently the organic phase was drained and collected separately in a 500 ml Erlenmeyer. The organic phase was dried over 8 g MgSO₄·2H₂O for 10 minutes. The mixture was filtered over a glass filter and the clear organic peroxide product 207 gram (assay 99%) was collected and stored at 20° C.

Analysis: Examples 5-8 were analyzed using the same method as is described for Examples 1 and 2 (“TBHP Protocol”).

| | TBHP (ppm) | TBHP (ppm) | TBHP (ppm) | ΔTBHP* (ppm) |
|---|---|---|---|---|
| | t = 0 weeks | t = 2 weeks | t = 4 weeks | 0-4 weeks |
| Example 5 | 8263 | 9140 | 9350 | 1087 |
| [Comparative] | | | | |
| Example 6 | 340 | 1017 | 1037 | 697 |
| Example 7 | 569 | 1068 | 1369 | 800 |
| Example 8 | 16 | 252 | 273 | 257 |

*A lower ΔTBHP value indicates increased storage stability

C. Ex. 5—pH in step c) always >6.8; no pH reduction after completion of step e)

Ex. 6—pH in step c)<6.8 before step d); no pH reduction after completion of step e)

Ex. 7—pH in step c)<6.8 before step d); no pH reduction after completion of step e)

Ex. 8—pH in step c)<6.8 before step d); pH reduction after completion of step e)

### Conclusions—Examples 1-8

These data show that having a pH of less than 6.8 in step c) before proceeding to step d) (“step i)”) reduces the hydroperoxide content in the product (>93% reduction at t=0) and increases storage stability (C.Ex.5 vs. Ex. 6/7). These data also demonstrate that reducing the pH following completion of step e) (“step ii)”) reduces the hydroperoxide content in the product (>86% reduction at t=0) and increases storage stability (Ex. 2 vs. Ex. 1; Ex. 4 vs. Ex. 3; Ex. 8 vs. Ex. 7).

The overall improvement obtained by combining steps i) and ii) is remarkable (>99% reduction in hydroperoxide content at t=0, substantial improvement in storage stability; C. Ex. 5 vs. Ex. 8). As shown above, this most typical process (Ex. 2, Ex. 4., Ex. 8) consistently generated t-butylperoxy products with an initial TBHP content of less than 300 ppm TBHP and a 4-week stability (ΔTBHP, 0-4 weeks) of less than 300 ppm.

The present disclosure may be further described by one or more of the following Aspects:

Aspect 1. A process for preparing a peroxyester or peroxycarbonate comprising:
a) reacting an organic hydroperoxide with an acid halide, an acid anhydride, or a haloformate, in the presence of a base,
b) separating the aqueous layer after completion of step a),
c) adding a reducing agent to the organic layer after the aqueous layer has been separated in step b), wherein the reducing agent is an agent capable of reducing the organic hydroperoxide to the corresponding alcohol,
d) ensuring that the mixture of step c) has a pH of greater than 6.8 by maintaining or increasing the pH of the mixture of step c), and
e) maintaining the pH of greater than 6.8 for at least 5 seconds,
wherein:
   i. the mixture of step c) has a pH of less than 6.8 before step d), and in step d) the pH of the mixture of step c) is increased to a pH of greater than 6.8; and/or
   ii. after completion of step e) the pH is decreased to a pH of less than 6.8.

Aspect 2. The process of Aspect 1, wherein the process further comprises increasing the pH to greater than 6.8 and/or washing the organic layer after completion of step ii), wherein the washing step(s) typically comprise one or more water washes and/or one or more alkaline aqueous washes, such as washing with NaOH(aq) and/or NaHCO₃(aq)).

Aspect 3. The process of Aspect 1 or 2, wherein the reducing agent added in step c) is a sulfite.

Aspect 4. The process of Aspect 3, wherein the sulfite is an aqueous sulfite solution.

Aspect 5. The process of any one of Aspects 1-4, wherein in step c) the pH is decreased to a pH of 6.5 or less.

Aspect 6. The process of any one of Aspects 1-5, wherein in step c) the pH is decreased to a pH of from about 4 to about 6.5.

Aspect 7. The process of any one of Aspects 1-6, wherein step d) comprises ensuring that the pH of the mixture of step c) is >7.0.

Aspect 8. The process of any one of Aspects 1-7, wherein step d) comprises ensuring that the pH of the mixture of step c) is >7.2.

Aspect 9. The process of any one of Aspects 1-8, wherein step d) comprises ensuring that the pH of the mixture of step c) is >7.4.

Aspect 10. The process of any one of Aspects 1-9, wherein step e) the pH is maintained for at least 10 seconds, typically at least 30 seconds, typically at least 60 seconds, and more typically at least 120 seconds.

Aspect 11. The process of any one of Aspects 2-10, wherein in step ii) the pH is decreased to a pH of about 6.5 or lower.

Aspect 12. The process of any one of Aspects 2-11, wherein in step ii) the pH is decreased to a pH of about 4.5 to 6.5.

Aspect 13. The process of any one of Aspects 1-12, wherein the organic hydroperoxide is selected from an organic hydroperoxide of the general formula (II):

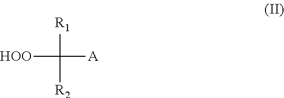

wherein:
   R₁ and R₂ are independently chosen from hydrogen, C₁-C₂₀ alkyl, C₃-C₂₀ cycloalkyl, C₆-C₂₀ aryl, C₇-C₂₀ aralkyl, and C₇-C₂₀ alkaryl, or R₁ and R₂ form a C₃-C₁₂ cycloalkyl group, which groups may include linear or branched alkyl moieties, and each of R₁ and R₂ may optionally be substituted with one or more groups selected from hydroxy, hydroperoxy, alkoxy, linear or branched alkyl, aryloxy, halogen, ester, carboxy, nitrile, and amido,
   A is selected independently of R₁ and R₂ from the same group of substituents as R₁ and R₂, or A is of the general formula (III):

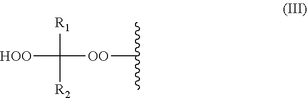

wherein R₁ and R₂ are as defined above, wherein A is typically selected independently of R₁ and R₂ from the same group of substituents as R₁ and R₂.

Aspect 14. The process of any one of Aspects 1-13, wherein the organic hydroperoxide is tert-butyl hydroperoxide, tert-amyl hydroperoxide, cumyl hydroperoxide, 1,1,3,3-tetramethylbutyl hydroperoxide, 4-hydroperoxy-4-methylpentan-2-ol, 2,5-dihydroperoxy-2,5-dimethylhex-3-yne, 2,5-dihydroperoxy-2,5-dimethylhexane, or mixtures thereof.

Aspect 15. The process of any one of Aspects 1-14, wherein the organic hydroperoxide is tert-butyl hydroperoxide, typically obtained from an air oxidation process.

Aspect 16. The process of any one of Aspects 1 to 15, wherein the acid halide, acid anhydride, or chloroformate is a reactive carbonyl compound of the general formulae (Ia) or (Ib):

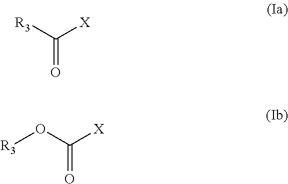

wherein:
   R₃ is independently chosen from C₁-C₂₀ alkyl, C₃-C₂₀ cycloalkyl, C₆-C₂₀ aryl, C₇-C₂₀ aralkyl, and C₇-C₂₀ alkaryl, wherein R₃ is optionally substituted with one or more groups selected from hydroxy, alkoxy, linear or branched alkyl, aryloxy, halogen, ester, carboxy, nitrile, and amido, and
   X is a halogen or —O—CO—R₃′, wherein R_{3T} is selected independently of R₃ from the same group of substituents as R₃.

Aspect 17. The process of any one of Aspects 1 to 16, wherein the acid halide, acid anhydride or haloformate is derived from any of the following carboxylic acids: acetic acid, phenylacetic acid, phenoxyacetic acid, propanoic acid, isobutyric acid, n-butyric acid, benzoic acid, 2-methyl-benzoic acid, 2-methylbutanoic acid, 2-butenoic acid, 3-phenylpropenic acid, 2,2-dimethylpropanoic acid, 2,2-dimethylbutanoic acid, 2,2-dimethylpentanoic acid, 2-ethylbutanoic acid, 3,5,5-trimethylhexanoic acid, 2-ethylhexanoic acid, neohexanoic acid, neoheptanoic acid, neodecanoic acid, octanoic acid, nonanoic acid, lauric acid, hexanedioic acid, 3,5,5-trimethylhexanedioic acid, 2,4,4-trimethylhexanedioic acid, decanedioic acid, undecanedioic acid, dodecanedioic acid, cyclohexanecarboxylic acid, 1,4-cyclohexanedicarboxylic acid, cyclohexane-1,4-diacetic acid, maleic acid, citric acid, methylsuccinic acid, citraconic acid, fumaric acid, oxalic acid, terephthalic acid, propenoic acid, and phthalic acid.

Aspect 18. The process of any one of Aspects 1 to 17, wherein the process uses an acid halide or a haloformate.

Aspect 19. The process of any one of Aspects 1 to 18, wherein the acid halide is an acid chloride.

Aspect 20. The process of Aspect 19, wherein the acyl portion of the acid chloride derives from any of the following carboxylic acids: acetic acid, phenylacetic acid, phenoxyacetic acid, propanoic acid, isobutyric acid, n-butyric acid, benzoic acid, 2-methyl-benzoic acid, 2-methylbutanoic acid, 2-butenoic acid, 3-phenylpropenic acid, 2,2-dimethylpropanoic acid, 2,2-dimethylbutanoic acid, 2,2-dimethylpentanoic acid, 2-ethylbutanoic acid, 3,5,5-trimethylhexanoic acid, 2-ethylhexanoic acid, neohexanoic acid, neoheptanoic acid, neodecanoic acid, octanoic acid, nonanoic acid, lauric acid, hexanedioic acid, 3,5,5-trimethylhexanedioic acid, 2,4,4-trimethylhexanedioic acid, decanedioic acid, undecanedioic acid, dodecanedioic acid, cyclohexanecarboxylic acid, 1,4-cyclohexanedicarboxylic acid, cyclohexane-1,4-diacetic acid, maleic acid, citric acid, methylsuccinic acid, citraconic acid, fumaric acid, oxalic acid, terephthalic acid, propenoic acid, and phthalic acid.

Aspect 21. The process of any one of Aspects 1 to 18, wherein the haloformate is a chloroformate.

Aspect 22. The process of Aspect 21, wherein the chloroformate is selected from 2-(1-methylethoxy)phenyl chloroformate, 1-methylpropyl chloroformate, 4-methylphenyl chloroformate, 2,2,2-trichloro-1,1-dimethylethyl chloroformate, heptyl chloroformate, cyclohexyl methyl chloroformate, ethylene glycol bis(chloroformate), 3-(1,1-dimethylethyl)phenyl chloroformate, 3-(trichlorosilyl)propyl chloroformate, phenyl chloroformate, 3-methoxybutyl chloroformate, 2-phenoxyethyl chloroformate, 2,2-dimethyl-1,3-propane diol bis(chloroformate), phenyl methyl chloroformate, 9-octadecenyl chloroformate, 2-methylphenyl chloroformate, bisphenol A bis(chloroformate), 1,3-dimethyl butyl chloroformate, 3,4-dimethyl butyl chloroformate, 3,4-dimethyl phenyl chloroformate, trichloromethyl chloroformate, 1-chloroethyl chloroformate, chloromethyl chloroformate, 1,4-butane diol bis(chloroformate), 1,1-bis (ethoxycarbo)ethyl chloroformate, 3,5-dimethyl phenyl chloroformate, octyl chloroformate, ethyl chloroformate, octadecyl chloroformate, (2-oxo-1,3-dioxolan-4-yl)methyl chloroformate, 1,6-hexane diol bis(chloroformate), 2-chlorobutyl chloroformate, 4-methoxyphenyl chloroformate, 2-methylpropyl chloroformate, 2-(methylsulfonyl)ethyl chloroformate, dodecyl chloroformate, 1,4-cyclohexane dimethanol bis(chloroformate), 2-chloro-2-phenyl ethyl chloroformate, 2-acryloyloxyethyl chloroformate, 4-nitrophenyl chloroformate, n-butyl chloroformate, decyl chloroformate, 2-ethylhexyl chloroformate, 2-propenyl chloroformate, 2-chlorocyclohexyl chloroformate, 2-methyl-2-propenyl chloroformate, cyclohexyl chloroformate, 2-chloroethyl chloroformate, [4-(phenylazo)phenyl]methyl chloroformate, hexadecyl chloroformate, 1-naphthalenyl chloroformate, 2-[2-cyclopentyl-4-(1,1-dimethylethyl)phenoxy]-1-methylethyl chloroformate, 3,5,5-trimethyl hexyl chloroformate, isotridecyl chloroformate, tridecyl chloroformate, 4-(1,1-dimethylethyl)cyclohexyl chloroformate, 2,4,5-trichlorophenyl chloroformate, 3-chloropropyl chloroformate, tetradecyl chloroformate, 9H-fluoren-9-yl methyl chloroformate, (4-nitrophenyl)methyl chloroformate, methyl chloroformate, 2-(1-methylethyl)phenyl chloroformate, triethylene glycol bis(chloroformate), 2-methoxyethyl chloroformate, 1-methylethenyl chloroformate, 3-methylphenyl chloroformate, 2-bromoethyl chloroformate, diethylene glycol bis(chloro-formate), 3-methyl-5-(1-methylethyl)phenyl chloroformate, 2,2,2-tribromoethyl chloroformate, 2-ethoxyethyl chloroformate, 3-methyl-1,5-pentane diol bis(chloroformate), 4-methoxy carbophenyl chloroformate, ethenyl chloroformate, 1-methylethyl chloroformate, 2-(1-methylpropyl)phenyl chloroformate, 2,2,2-trichloroethyl chloroformate, pentyl chloroformate, cyclodecyl chloroformate, 4-(1,1-dimethylethyl)phenyl chloroformate, hexyl chloroformate, n-propyl chloroformate, 3-methoxy-3-methylbutyl chloroformate, 2-propoxyethyl chloroformate, 2-methoxy-1-methylethyl chloroformate, 2-butoxyethyl chloroformate, 2,2-dimethyl propyl chloroformate, 2,3-dihydro-2,2-dimethyl-7-benzofuranyl chloroformate, 1-chloroethyl chloroformate, cyclobutyl chloroformate, 5-methyl-2-(1-methylethyl)cyclohexyl chloroformate, 1,1-dimethyl ethyl chloroformate, 1-methylheptyl chloroformate, and mixtures thereof.

Aspect 23. The process of any one of Aspects 1 to 22, wherein the base is selected from sodium carbonate, potassium carbonate, sodium hydroxide, potassium hydroxide, and mixtures thereof.

Aspect 24. The process of any one of Aspects 1 to 23, wherein in any one of steps c)-e) and ii) the mixture is agitated for at least 1 minute before proceeding to the next step of the process.

Aspect 25. A tert-butyl peroxyester or a tert-butyl peroxycarbonate, exemplified in that the tert-butyl peroxyester or tert-butyl peroxycarbonate has an initial (t=0) tert-butylhydroperoxide (TBHP) content of less than 300 ppm and a four-week stability (0-4 week ΔTBHP) value of 300 ppm or less, wherein the initial TBHP content and the four-week stability value are determined in accordance with the “TBHP Protocol”.

Aspect 26. A tert-butyl peroxyester, exemplified in that the tert-butyl peroxyester has an initial (t=0) tert-butylhydroperoxide (TBHP) content of less than 300 ppm and a four-week stability (0-4 week ΔTBHP) value of 300 ppm or less, wherein the initial TBHP content and the four-week stability value are determined in accordance with the “TBHP Protocol”.

Aspect 27. The tert-butyl peroxyester of Aspect 26, wherein the tert-butyl peroxyester is tert-butyl peroxy-3,5,5-trimethyl hexanoate (CAS: 13122-18-4), tert-Butyl peroxy-2-ethylhexanoate (CAS: 3006-82-4), or tert-Butyl peroxybenzoate (CAS: 614-45-9).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the present disclosure in any way. Rather, the foregoing detailed description will provide those skilled in the art with a convenient road map for implementing an exemplary embodiment as contemplated herein. It should be understood that various changes may be made in the function and arrangement of elements described in an exemplary embodiment without departing from the scope of the present disclosure as set forth in the appended claims.

What is claimed is:

1. A process for preparing a peroxyester or peroxycarbonate comprising:
a) reacting an organic hydroperoxide with an acid halide, an acid anhydride, or a haloformate, in the presence of a base to form an aqueous layer and an organic layer,
b) separating the aqueous layer from the organic layer after completion of step a),
c) adding a reducing agent to the organic layer after the aqueous layer has been separated from the organic layer in step b), wherein the reducing agent reduces the organic hydroperoxide to a corresponding alcohol and forms a mixture,
d) maintaining or adjusting the pH of the mixture of step c) to greater than about 6.8, and
e) maintaining the pH of the mixture of greater than about 6.8 for at least 5 seconds,
wherein
i. the mixture of step c) has a pH of less than about 6.8 before step d), and in step d) the pH of the mixture of step c) is increased to a pH of greater than about 6.8; and/or
ii. after completion of step e) the pH of the mixture is decreased to a pH of less than about 6.8.

2. The process of claim 1, wherein the reducing agent added in step c) is a sulfite.

3. The process of claim 2, wherein the sulfite is an aqueous sulfite solution.

4. The process of claim 1, wherein in step i) the mixture of step c) has a pH of about 6.5 or less.

5. The process of claim 1, wherein in step i) the mixture of step c) has a pH of from about 4 to about 6.5.

6. The process of claim 1, wherein the pH of the mixture in step d) is greater than about 7.0.

7. The process of claim 1, wherein in step ii) the pH is decreased to a pH of about 6.5 or lower.

8. The process of claim 1, wherein the organic hydroperoxide is an organic hydroperoxide of the general formula (II): 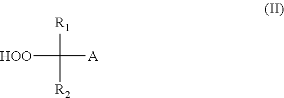
wherein:
each of R₁ and R₂ is independently chosen from hydrogen, C₁-C₂₀ alkyl, C₃-C₂₀ cycloalkyl, C₆-C₂₀ aryl, C₇-C₂₀ aralkyl, and C₇-C₂₀ alkaryl, or
each of R₁ and R₂ is independently formed from a C₃-C₁₂ cycloalkyl group and each of R₁ and R₂ is independently optionally substituted with one or more groups chosen from hydroxy, hydroperoxy, alkoxy, linear or branched alkyl, aryloxy, halogen, ester, carboxy, nitrile, and amido groups,
A is selected independently of R₁ and R₂ from the same group of substituents as R₁ and R₂, or A is of the general formula (III): 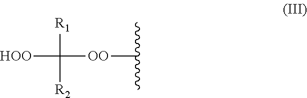
wherein R₁ and R₂ are as defined above.

9. The process of claim 1, wherein the organic hydroperoxide is tert-butyl hydroperoxide, tert-amyl hydroperoxide, cumyl hydroperoxide, 1,1,3,3-tetramethylbutyl hydroperoxide, 4-hydroperoxy-4-methylpentan-2-ol, 2,5-dihydroperoxy-2,5-dimethylhex-3-yne, 2,5-dihydroperoxy-2,5-dimethylhexane, or mixtures thereof.

10. The process of claim 1, wherein the organic hydroperoxide is tert-butyl hydroperoxide.

11. The process of claim 1, wherein the acid halide, acid anhydride, or haloformate is a reactive carbonyl compound of the general formulae (Ia) or (Ib): 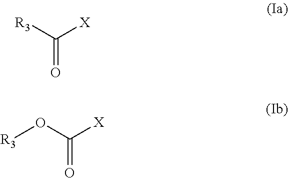
wherein:
R₃ is chosen from C₁-C₂₀ alkyl, C₃-C₂₀ cycloalkyl, C₆-C₂₀ aryl, C₇-C₂₀ aralkyl, and C₇-C₂₀ alkaryl groups, wherein R₃ is optionally substituted with one or more groups chosen from hydroxy, alkoxy, linear or branched alkyl, aryloxy, halogen, ester, carboxy, nitrile, and amido groups, and
X is a halogen or —O—CO—R_{3′}, wherein R₃, is selected independently of R₃ and is chosen from the same group of substituents as R₃ as defined above.

12. The process of claim 1, wherein the acid halide, acid anhydride or haloformate is derived from acetic acid, phenylacetic acid, phenoxyacetic acid, propanoic acid, isobutyric acid, n-butyric acid, benzoic acid, 2-methyl-benzoic acid, 2-methylbutanoic acid, 2-butenoic acid, 3-phenylpropenic acid, 2,2-dimethylpropanoic acid, 2,2-dimethylbutanoic acid, 2,2-dimethylpentanoic acid, 2-ethylbutanoic acid, 3,5,5-trimethylhexanoic acid, 2-ethylhexanoic acid, neohexanoic acid, neoheptanoic acid, neodecanoic acid, octanoic acid, nonanoic acid, lauric acid, 3,5,5-trimethylpentanedioic acid, hexanedioic acid, 3,5,5-trimethylhexanedioic acid, 2,4,4-trimethylhexanedioic acid, decanedioic acid, undecanedioic acid, dodecanedioic acid, cyclohexanecarboxylic acid, 1,4-cyclohexanedicarboxylic acid, cyclohexane-1,4-diacetic acid, maleic acid, citric acid, methylsuccinic acid, citraconic acid, fumaric acid, oxalic acid, terephthalic acid, propenoic acid, or phthalic acid.

13. The process of claim 1, wherein step a) utilizes the acid halide and/or the haloformate.

14. The process of claim 13, wherein the acid halide is an acid chloride comprising an acryl portion that corresponds to an acyl portion of any one of the following carboxylic acids: acetic acid, phenylacetic acid, phenoxyacetic acid, propanoic acid, isobutyric acid, n-butyric acid, benzoic acid, 2-methyl-benzoic acid, 2-methylbutanoic acid, 2-butenoic acid, 3-phenylpropenic acid, 2,2-dimethylpropanoic acid, 2,2-dimethylbutanoic acid, 2,2-dimethylpentanoic acid, 2-ethylbutanoic acid, 3,5,5-trimethylhexanoic acid, 2-ethylhexanoic acid, neohexanoic acid, neoheptanoic acid, neodecanoic acid, octanoic acid, nonanoic acid, lauric acid, 3,5,5-trimethylpentanedioic acid, hexanedioic acid, 3,5,5-trimethylhexanedioic acid, 2,4,4-trimethylhexanedioic acid, decanedioic acid, undecanedioic acid, dodecanedioic acid, cyclohexanecarboxylic acid, 1,4-cyclohexanedicarboxylic acid, cyclohexane-1,4-diacetic acid, maleic acid, citric acid, methylsuccinic acid, citraconic acid, fumaric acid, oxalic acid, terephthalic acid, propenoic acid, and phthalic acid; and/or
wherein the haloformate is a chloroformate and is chosen from 2-(1-methylethoxy)phenyl chloroformate, 1-methylpropyl chloroformate, 4-methylphenyl chloroformate, 2,2,2-trichloro-1,1-dimethylethyl chloroformate, heptyl chloroformate, cyclohexyl methyl chloroformate, ethylene glycol bis(chloroformate), 3-(1,1-dimethylethyl)phenyl chloroformate, 3-(trichlorosilyl)propyl chloroformate, phenyl chloroformate, 3-methoxybutyl chloroformate, 2-phenoxyethyl chloroformate, 2,2-dimethyl-1,3-propane diol bis(chloroformate), phenyl methyl chloroformate, 9-octadecenyl chloroformate, 2-methylphenyl chloroformate, bisphenol A bis(chloroformate), 1,3-dimethyl butyl chloroformate, 3,4-dimethyl butyl chloroformate, 3,4-dimethyl phenyl chloroformate, trichloromethyl chloroformate, 1-chloroethyl chloroformate, chloromethyl chloroformate, 1,4-butane diol bis(chloroformate), 1,1-bis (ethoxycarbo)ethyl chloroformate, 3,5-dimethyl phenyl chloroformate, octyl chloroformate, ethyl chloroformate, octadecyl chloroformate, (2-oxo-1,3-dioxolan-4-yl)methyl chloroformate, 1,6-hexane diol bis(chloroformate), 2-chlorobutyl chloroformate, 4-methoxyphenyl chloroformate, 2-methylpropyl chloroformate, 2-(methylsulfonyl)ethyl chloroformate, dodecyl chloroformate, 1,4-cyclohexane dimethanol bis(chloroformate), 2-chloro-2-phenyl ethyl chloroformate, 2-acryloyloxyethyl chloroformate, 4-nitrophenyl chloroformate, n-butyl chloroformate, decyl chloroformate, 2-ethylhexyl chloroformate, 2-propenyl chloroformate, 2-chlorocyclohexyl chloroformate, 2-methyl-2-propenyl chloroformate, cyclohexyl chloroformate, 2-chloroethyl chloroformate, [4-(phenylazo)phenyl]methyl chloroformate, hexadecyl chloroformate, 1-naphthalenyl chloroformate, 2-[2-cyclopentyl-4-(1,1-dimethylethyl)phenoxy]-1-methylethyl chloroformate, 3,5,5-trimethyl hexyl chloroformate, isotridecyl chloroformate, tridecyl chloroformate, 4-(1,1-dimethylethyl)cyclohexyl chloroformate, 2,4,5-trichlorophenyl chloroformate, 3-chloropropyl chloroformate, tetradecyl chloroformate, 9H-fluoren-9-yl methyl chloroformate, (4-nitrophenyl)methyl chloroformate, methyl chloroformate, 2-(1-methylethyl)phenyl chloroformate, triethylene glycol bis(chloroformate), 2-methoxyethyl chloroformate, 1-methylethenyl chloroformate, 3-methylphenyl chloroformate, 2-bromoethyl chloroformate, diethylene glycol bis(chloro-formate), 3-methyl-5-(1-methylethyl)phenyl chloroformate, 2,2,2-tribromoethyl chloroformate, 2-ethoxyethyl chloroformate, 3-methyl-1,5-pentane diol bis(chloroformate), 4-methoxy carbophenyl chloroformate, ethenyl chloroformate, 1-methylethyl chloroformate, 2-(1-methylpropyl)phenyl chloroformate, 2,2,2-trichloroethyl chloroformate, pentyl chloroformate, cyclodecyl chloroformate, 4-(1,1-dimethylethyl)phenyl chloroformate, hexyl chloroformate, n-propyl chloroformate, 3-methoxy-3-methylbutyl chloroformate, 2-propoxyethyl chloroformate, 2-methoxy-1-methylethyl chloroformate, 2-butoxyethyl chloroformate, 2,2-dimethyl propyl chloroformate, 2,3-dihydro-2,2-dimethyl-7-benzofuranyl chloroformate, 1-chloroethyl chloroformate, cyclobutyl chloroformate, 5-methyl-2-(1-methylethyl)cyclohexyl chloroformate, 1,1-dimethyl ethyl chloroformate, 1-methylheptyl chloroformate, and mixtures thereof.

15. The process of claim 1 wherein:
the reducing agent added in step c) is an aqueous sulfite solution;
in step i) the mixture of step c) has a pH of from about 4 to about 6.5;
the pH of the mixture in step d) is greater than about 7.0; and
in step ii) the pH is decreased to a pH of about 6.5 or lower.

16. The process of claim 15 wherein:
the organic hydroperoxide is tert-butyl hydroperoxide; and
the acid halide is an acid chloride and/or the haloformate is a chloroformate.

17. A tert-butylperoxy ester or a tert-butylperoxy carbonate formed from the process of claim 1 and having an initial (t=0 weeks) tert-butylhydroperoxide content of less than 300 ppm and a four-week stability (0-4 weeks Δ t-Bu-OOH) value of 300 ppm or less.
